(19) United States
(12) Patent Application Publication
(10) Pub. No.: US 2022/0125730 A1
(43) Pub. Date: Apr. 28, 2022

(54) METHOD OF MANUFACTURING AN ORALLY DISINTIGRATING TABLET

(71) Applicant: DEXCEL PHARMA TECHNOLOGIES LTD., Or-Akiva (IL)

(72) Inventors: Sheera MOSES-HELLER, Atlit (IL); Lihi BLESSER, Binyamina (IL)

(73) Assignee: DEXCEL PHARMA TECHNOLOGIES LTD., Or-Akiva (IL)

(21) Appl. No.: 17/570,548

(22) Filed: Jan. 7, 2022

Related U.S. Application Data
(63) Continuation of application No. 15/801,886, filed on Nov. 2, 2017.

(51) Int. Cl. A61K 9/16; A61K 9/00; A61P 1/04; A61K 31/4439; A61K 9/20

(52) U.S. Cl. CPC A61K 9/1676; A61K 9/0056; A61P 1/04; A61K 31/4439; A61K 9/20; A61K 9/1623; A61K 9/1611; A61K 9/1617; A61K 9/1652; A61K 9/1635

(57) ABSTRACT

An orally disintegrating composition having a bimodal particle size distribution, methods for its production and use thereof are provided.

### CROSS REFERENCE TO RELATED APPLICATION(S)

This is a continuation Application of U.S. application Ser. No. 15/801,886 filed on Nov. 2, 2017. The entirety of the disclosure of the above-referenced application is incorporated herein by reference.

### TECHNICAL FIELD

A multiparticulate composition comprising a proton pump inhibitor, methods for its production and use thereof are provided.

### BACKGROUND

Proton pump inhibitors (PPIs) are typically benzimidazole derivatives which inhibit the H⁺/K⁺-ATPase enzyme system at the secretory surface of the gastric parietal cells thereby inhibiting gastric acid secretion. Compositions comprising PPIs are typically designed for oral administration as single dosage forms such as tablets, capsules and sachet. When compressed into tablets, these compositions may be produced as a single unit form or a multiunit system comprising a plurality of particles, each containing the active ingredient. Due to the acid-liability of PPIs, compositions comprising same are typically formulated with an enteric coating layer designed to protect the active ingredient during passage through the acidic environment of the stomach. In a multiunit system, each particle containing the active ingredient is coated with an enteric layer over the active ingredient to afford its protection.

Orally disintegrating compositions or orodisperse formulations are dosage forms which disintegrate in the oral cavity into small particles, suitable for being swallowed, even without the need for fluids. These compositions provide improved patient compliance, particularly in patients who experience difficulties swallowing conventional dosage forms such as pediatric and geriatric patients, subjects who suffer from impaired swallowing and subjects who suffer from psychiatric disorders.

Orally disintegrating compositions comprising PPIs as active ingredients have been described in U.S. Pat. Nos. 6,586,004; 7,399,485; 7,431,942; 7,838,033; 8,486,450; 8,545,881; 8,715,730; 9,060,936; 9,198,862; 9,241,910; 9,486,446; 9,526,789; and U.S. patent application publication numbers 2008/0305166; 2011/0229570; 2012/0282335; 2013/0202688; 2013/273157; and 2015/0272889.

U.S. 2016/0354356 discloses an orally disintegrating dosage form of a proton pump inhibitor, methods for its production and use thereof. The dosage form includes a plurality of pellets containing a proton pump inhibitor admixed with a disintegrant to afford rapid disintegration in the oral cavity after administration.

There remains an unmet need for orally disintegrating compositions which afford improved patient compliance.

### SUMMARY

The present disclosure relates to an orally disintegrating composition comprising a therapeutically effective amount of a PPI. The composition comprises two distinct populations of particles comprising a bimodal particle size distribution. The composition provides improved patient compliance.

According to a first aspect, there is provided an orally disintegrating composition comprising a plurality of particles having a bimodal size distribution comprising a first population of particles comprising a proton pump inhibitor and a second population of particles comprising a disintegrant, wherein the ratio of the median particle size of the first population of particles to the median particle size of the second population of particles is about 2:1 to about 5:1. Surprisingly, the ratio of the median particle size of the first population of particles to the median particle size of the second population of particles as defined herein is critical to obtain the unexpectedly advantageous properties of the claimed invention.

In one embodiment, the first population of particles has a median particle size in the range of about 400 μm to about 600 μm. In other embodiments, the first population of particles has a median particle size in the range of about 450 μm to about 550 μm. In some embodiments, the first population of particles has a d₁₀ particle size in the range of about 300 μm to about 500 μm. In other embodiments, the first population of particles has a d₁₀ particle size in the range of about 350 μm to about 450 μm. In various embodiments, the first population of particles has a d₉₀ particle size in the range of about 500 μm to about 700 μm. In other embodiments, the first population of particles has a d₉₀ particle size in the range of about 550 μm to about 650 μm.

In some embodiments, the second population of particles has a median particle size in the range of about 50 μm to about 250 μm. In other embodiments, the second population of particles has a median particle size in the range of about 100 μm to about 200 μm. In further embodiments, the second population of particles has a d₁₀ particle size in the range of about 1 μm to about 100 μm. In additional embodiments, the second population of particles has a d₁₀ particle size in the range of about 5 μm to about 50 μm. In one embodiment, the second population of particles has a d₉₀ particle size in the range of about 250 μm to about 500 μm. In other embodiments, the second population of particles has a d₉₀ particle size in the range of about 300 μm to about 450 μm.

In various embodiments, the weight percent ratio of the first population of particles to the second population of particles is about 0.1:1 to about 1:0.1. In some embodiments, the weight percent ratio of the first population of particles to the second population of particles is about 0.5:1 to about 1:0.5. In one embodiment, the weight percent ratio of the first population of particles to the second population of particles is about 0.5:1. In another embodiment, the weight percent ratio of the first population of particles to the second population of particles is about 1:1. In further embodiments, the weight percent ratio of the first population of particles to the second population of particles is about 1:0.8. In additional embodiments, the weight percent ratio of the first population of particles to the second population of particles is about 1:0.5. Surprisingly, the weight percent ratio of the first population of particles to the second population of particles as defined herein is critical to obtain the unexpectedly advantageous properties of the claimed invention.

In certain embodiments, the orally disintegrating composition is an orally disintegrating tablet.

In several embodiments, the PPI comprises lansoprazole, omeprazole, pantoprazole, leminoprazole, perprazole, rabeprazole, or a pharmaceutically acceptable salt thereof, and a mixture or combination thereof.

It is contemplated that any pharmaceutically acceptable form of the PPI including, but not limited to, salts (e.g. alkaline salts), solvates (e.g. hydrates), isomers, isomorphs, polymorphs, pseudopolymorphs, and prodrugs thereof are within the scope of the present disclosure.

In particular embodiments, the PPI comprises lansoprazole or a pharmaceutically acceptable salt thereof.

In one embodiment, the first population of particles comprises a plurality of cores comprising inert seeds coated a therapeutically effective amount of a PPI. In another embodiment, the inert seeds are in the form of granules, pellets, beads, or powder, and a mixture or combination thereof. Each possibility represents a separate embodiment. In certain embodiments, the inert seeds comprise sugar spheres. In other embodiments, the inert seeds comprise microcrystalline cellulose particles.

In some embodiments, the plurality of cores further comprise at least one of a binder, a filler, a surfactant, and an alkalizing agent. Each possibility represents a separate embodiment.

In various embodiments, the plurality of cores are coated with an enteric coating comprising one or more enteric polymers. In certain embodiments, the one or more enteric polymers are selected from the group consisting of hydroxypropyl methylcellulose phthalate (HPMCP), hydroxypropyl methylcellulose acetate succinate (HPMCAS), polyvinyl acetate phthalate, cellulose acetate trimellitate, cellulose acetate phthalate (CAP), polymethacrylic acid, polymethyl methacrylate, polyethyl methacrylate, and a mixture or combination thereof, with each possibility representing a separate embodiment. In one embodiment, the enteric polymer is hydroxypropyl methylcellulose phthalate. In another embodiment, the enteric polymer is hydroxypropyl methylcellulose acetate succinate. In some embodiments, the enteric coating further comprises at least one of a plasticizer, a colorant, a glidant, and an anti-tacking agent. Each possibility represents a separate embodiment.

In certain aspects and embodiments, the plurality of cores are further coated with a subcoating layer over the cores, to protect the enteric coating, once applied, from reacting with the alkaline cores containing the PPI. In some embodiments, the subcoating layer comprises at least one of hydroxypropyl methylcellulose, hydroxypropyl cellulose, polyvinylpyrrolidone, polyethylene glycol, and polyvinyl alcohol, with each possibility representing a separate embodiment. In further embodiments, the subcoating layer further comprises at least one of a filler, a surfactant, and an anti-tacking agent. Each possibility represents a separate embodiment.

In additional embodiments, the enteric coated cores are overcoated with a taste-masking coating.

According to certain aspects and embodiments, the first population of particles comprises:
(a) inert seeds;
(b) a drug layer over the inert seeds, the drug layer comprising a therapeutically effective amount of a PPI, a binder, a filler, a surfactant, and an alkalizing agent;
(c) a subcoating layer over the drug layer, the subcoating layer comprising a binder, a filler, a surfactant, and an anti-tacking agent; and
(d) an enteric coating over the subcoating layer, the enteric coating comprising an enteric polymer, a plasticizer, a colorant, and optionally a glidant, and an anti-tacking agent.

In certain embodiments, the disintegrant in the second population of particles is selected from the group consisting of crospovidone, croscarmellose sodium, a cellulose derivative, cross-linked derivatives of starch, pregelatinized starch, crosslinked sodium carboxymethyl cellulose, low substituted hydroxypropyl cellulose, and a mixture or combination thereof. Each possibility represents a separate embodiment.

The orally disintegrating pharmaceutical composition of the present disclosure is useful for inhibiting gastric acid secretion in the treatment of gastroesophageal reflux disease, gastritis, peptic ulcers (duodenal and gastric), and erosive esophagitis, with each possibility representing a separate embodiment.

Accordingly, there is provided a method of inhibiting gastric acid secretion, the method comprising administering to a subject in need thereof the orally disintegrating composition disclosed herein. In other embodiments, there is provided a method of treating a disease or disorder selected from the group consisting of gastroesophageal reflux disease, gastritis, peptic ulcers (duodenal and gastric), and erosive esophagitis, the method comprising administering to a subject in need thereof the orally disintegrating composition disclosed herein.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DETAILED DESCRIPTION

The following paragraphs describe in more detail the embodiments of the invention disclosed herein. The following embodiments are not meant to limit the invention or narrow the scope thereof, as it will be readily apparent to one of ordinary skill in the art that suitable modifications and adaptations may be made without departing from the scope of the invention, embodiments, or specific aspects described herein. All patents and publications cited herein are incorporated by reference in their entirety.

Described herein is an orally disintegrating pharmaceutical composition. In certain aspects and embodiments, the pharmaceutical composition is in the form of an orally disintegrating tablet (ODT). The term “orally disintegrating tablet” as used herein refers to a tablet which substantially disintegrates in an oral cavity of a subject in need thereof within about 60 seconds or less after administration. In one embodiment, disintegration is measured in vitro using e.g. the USP <701> Disintegration Test. In another embodiment, “orally disintegrating tablet” refers to a loss of structural integrity of the tablet following administration to the buccal cavity of a subject when in contact with the mucosal tissue of the tongue, cheek, and/or mouth. The orally disintegrating tablet is typically placed on the tongue (lingual administration) which stimulates saliva generation and enhances disintegration of the composition. Following disintegration, a suspension of undissolved particles in saliva is typically formed. The particles can then be swallowed, even without the need for water or other fluids, allowing for absorption of the active pharmaceutical ingredient in the GI tract, generally in the upper intestine.

According to certain aspects and embodiments, the orally disintegrating composition comprises a plurality of particles having a bimodal size distribution comprising a first population of particles comprising a PPI and a second population of particles comprising a disintegrant, wherein the ratio of the median particle size of the first population to the median particle size of the second population is about 2:1 to about 5:1, including all iterations of ratios between the specified range. In one embodiment, the ratio of the median particle size of the first population to the median particle size of the second population is about 2.5:1 to about 5:1, including all iterations of ratios between the specified range. In one embodiment, the ratio of the median particle size of the first population to the median particle size of the second population is about 2:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.9:1, about 4:1, about 4.1:1, about 4.2:1, about 4.3:1, about 4.4:1, about 4.5:1, about 4.6:1, about 4.7:1, about 4.8:1, about 4.9:1, or about 5:1, with each possibility representing a separate embodiment. The second population is devoid of PPI.

The orally disintegrating composition disclosed herein affords high compliance by end-users. Furthermore, the bimodal size distribution provides improved compressibility and processability with superior physical properties (e.g. friability) of the obtained composition during packaging, transport and storage.

The term “bimodal” as used herein refers to a particle size distribution that comprises two components designated first and second populations. In some embodiments, the term “bimodal” refers to a particle size distribution that can be deconvoluted into two components having varying degrees of separation. It is contemplated that in some embodiments a bimodal size distribution may be deconvoluted to fit more than two peaks or that one component may exhibit a shoulder or tail.

The orally disintegrating composition of the disclosure is described in more details below:

1^{st} Population of Particles

The orally disintegrating composition described herein comprises a first population of particles comprising a therapeutically effective amount of a PPI.

According to certain aspects and embodiments, the first population of particles has a median particle size in the range of about 400 μm to about 600 μm, including each integer within the specified range. In some embodiments, the first population of particles has a median particle size in the range of about 450 μm to about 550 μm, including each integer within the specified range. In various embodiments, the first population of particles has a median particle size of about 400 μm, about 420 μm, about 440 μm, about 460 μm, about 480 μm, about 500 μm, about 520 μm, about 540 μm, about 560 μm, about 580 μm, or about 600 μm, with each possibility representing a separate embodiment. In further embodiments, the first population of particles has a d₁₀ particle size in the range of about 300 μm to about 500 μm, including each integer within the specified range. In additional embodiments, the first population of particles has a d₁₀ particle size in the range of about 350 μm to about 450 μm, including each integer within the specified range. In various embodiments, the first population of particles has a d₉₀ particle size in the range of about 500 μm to about 700 μm, including each integer within the specified range. In other embodiments, the first population of particles has a d₉₀ particle size in the range of about 550 μm to about 650 μm, including each integer within the specified range.

The terms “median” or “d₅₀” as used herein refer a particle diameter value in which the cumulative distribution percentage reaches 50%. In other words, the d₅₀ particle diameter or median particle size represents a value where half the particle population has particle diameters smaller than this value and half the particle population has particle diameters larger than this value. Similarly, the term “d₁₀” as used herein refers to a value where 10% of the particle population has particle diameters smaller than this value and 90% of the particle population has particle diameters larger than this value. Likewise, the term “d₉₀” as used herein refers to a value where 90% of the particle population has particle diameters smaller than this value and 10% of the particle population has particle diameters larger than this value. Particle size distribution can be measured using various techniques known in that art including, but not limited to, laser diffraction, light scattering, sedimentation field flow fractionation, photon correlation spectroscopy, disc centrifugation, and the like.

PPIs, as used herein, refer to any pharmacologically active ingredient which inhibits the hydrogen potassium adenosine triphosphatase enzyme system (e.g., the H⁺/K⁺-ATPase) of gastric parietal cells. As described herein, PPIs may include benzimidazole derivatives, imidazopyridine derivatives or a potassium-competitive inhibitor and mixtures thereof. Each possibility represents a separate embodiment. It is contemplated that the inhibition by the PPI may be irreversible or reversible. Exemplary benzimidazole derivative PPIs include, but are not limited to, lansoprazole, dexlansoprazole, omeprazole, esomeprazole, pantoprazole, rabeprazole, ilaprazole and AGN201904; each possibility represents a separate embodiment. Exemplary imidazopyridine derivative PPIs include, but are not limited to, tenatoprazole; and exemplary potassium-competitive inhibitors include, but are not limited to, revaprazan. In one embodiment, the PPI is lansoprazole.

According to the principles provided herein, PPIs include alkali metal salts and/or alkaline earth metal salts thereof such as, but not limited to, sodium, potassium, calcium, and magnesium salts. Each possibility represents a separate embodiment. PPIs may also be in the form of pharmaceutically acceptable uncharged or charged molecules, molecular complexes, solvates (including hydrates), or anhydrates thereof, single isomers including single enantiomers, racemates, or mixtures thereof; and any of the crystalline or amorphous forms.

In some embodiments, the PPI is in an amount of about 1% to about 15% by weight of the total mass of the orally disintegrating composition, including each integer within the specified range. The total orally disintegrating composition mass, as used herein, refers to the combined weight of the first and second populations as described herein. In other embodiments, the PPI is in an amount of about 2% to about 10% by weight of the total mass of the orally disintegrating composition, including each integer within the specified range. In yet other embodiments, the PPI is in an amount of about 3% to about 9% by weight of the total mass of the orally disintegrating composition, including each integer within the specified range. In various embodiments, the PPI is in an amount of about 1%, about 2%, about 3%, about 4%, about 5%, about 6%, about 7%, about 8%, about 9%, about 10%, about 11%, about 12%, about 13%, about 14%, or about 15% by weight of the total mass of the orally disintegrating composition, with each possibility representing a separate embodiment.

In certain embodiments, the first population of particles comprise a plurality of cores comprising a therapeutically effective amount of a PPI. In one embodiment, the cores are in the form of granules, pellets, beads, powder, and a mixture or combination thereof. Each possibility represents a separate embodiment. The cores typically comprise the PPI and one or more pharmaceutically acceptable excipients (e.g. a filler, a binder, an alkalizing agent etc.). In one embodiment, the cores, also referred to as “active cores”, comprise a matrix in which the PPI is embedded in one or more pharmaceutically acceptable excipients. These types of cores may be generated through methods well-known in the pharmaceutical arts, for example, dry or wet granulation, extrusion or spheronization.

In another embodiment, the cores comprise inert seeds coated with a drug layer comprising the PPI and one or more pharmaceutically acceptable excipients (e.g. a filler, a binder, an alkalizing agent etc.). Suitable inert seeds may be any pharmaceutically acceptable filler in the form of a bead or pellet (e.g., made of sugar or microcrystalline cellulose (MCC)), a granule, a powder or other seeds known in the art which may be spherical or semispherical in shape. Exemplary and non-limiting inert seeds onto which the drug-containing layer is applied are usually comprised of sugars, starch or cellulosic materials or combinations thereof, for example sugar derivatives such as lactose, sucrose, hydrolyzed starch (maltodextrins) or celluloses or mixtures thereof. In one embodiment, the inert seeds comprise a blend of starch and sugar. These inert seeds, also called nonpareils or sugar spheres, typically comprise spheres composed of sucrose and starch (for example, maize starch). In another embodiment, the inert seeds comprise microcrystalline cellulose particles. Other types of seeds may also be used.

In some embodiments, the inert seeds are in an amount of about 2% to about 20% by weight of the total mass of the orally disintegrating composition, including each integer within the specified range. In other embodiments, the inert seeds are in an amount of about 2% to about 15% by weight of the total mass of the orally disintegrating composition, including each integer within the specified range. In yet other embodiments, the inert seeds are in an amount of about 2% to about 10% by weight of the total mass of the orally disintegrating composition, including each integer within the specified range. In various embodiments, the inert seeds are in an amount of about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of the total mass of the orally disintegrating composition, with each possibility representing a separate embodiment.

In some embodiments, the plurality of cores further comprises an excipient such as, but not limited to, a binder, a filler, a surfactant, an alkalizing agent, and a mixture or combination thereof, which is either embedded in the matrix of the active cores or present as part of the drug layer. Suitable binders include, but are not limited to, polyvinylpyrrolidone (povidone), copovidone, hydroxypropylmethyl cellulose, starch, gelatin, or sugars. Sugars include sucrose, dextrose, molasses, and lactose. Each possibility represents a separate embodiment. In one embodiment, the binder in the cores is in an amount of about 0.01% to about 8% by weight of the total mass of the orally disintegrating composition, including each integer within the specified range. In several embodiments, the binder in the cores is in an amount of about 0.1% to about 5% by weight of the total mass of the orally disintegrating composition, including each integer within the specified range. In additional embodiments, the binder in the cores is in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, or about 8% by weight of the total mass of the orally disintegrating composition, with each possibility representing a separate embodiment. In certain embodiments, the binder in the cores comprises hydroxypropyl methyl cellulose (HPMC). In other embodiments, the binder in the cores comprises povidone or copovidone.

Suitable fillers include, but are not limited to, sugars such as lactose, sucrose, mannitol or sorbitol and derivatives therefore (e.g. amino sugars), ethylcellulose, microcrystalline cellulose, silicified microcrystalline cellulose and the like. Each possibility represents a separate embodiment. In certain embodiments, the filler in the cores is in an amount of about 0% to about 10% by weight of the total mass of the orally disintegrating composition, including each integer within the specified range. In several embodiments, the filler in the cores is in an amount of about 0.1% to about 7% by weight of the total mass of the orally disintegrating composition, including each integer within the specified range. In additional embodiments, the filler in the cores is in an amount of about 0%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the total mass of the orally disintegrating composition, with each possibility representing a separate embodiment. In other embodiments, the filler in the cores comprises mannitol.

Suitable surfactants include, but are not limited to, non-ionic, anionic or cationic surfactants. Typically, surfactants may have one lipophilic and one hydrophilic group in the molecule. The surfactant may optionally comprise one or more of soaps, detergents, emulsifiers, dispersing and wetting agents. More specifically, surfactants may optionally comprise, for example, one or more of polysorbate, stearyltriethanolamine, sodium lauryl sulfate, sodium taurocholate, laurylaminopropionic acid, lecithin, benzalkonium chloride, benzethonium chloride and glycerin monostearate among others. Each possibility represents a separate embodiment. In various embodiments, the surfactant in the cores is in an amount of about 0% to about 8% by weight of the total mass of the orally disintegrating composition, including each integer within the specified range. In several embodiments, the surfactant in the cores is in an amount of about 0.01% to about 5% by weight of the total mass of the orally disintegrating composition, including each integer within the specified range. In additional embodiments, the surfactant in the cores is in an amount of about 0%, about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, or about 8% by weight of total mass of the orally disintegrating composition, with each possibility representing a separate embodiment. In certain embodiments, the surfactant in the cores comprises polysorbate (e.g. Tween-80).

Suitable alkalizing agents include, but are not limited to, organic and inorganic alkaline substances. Suitable organic alkaline substances include, but are not limited to, basic amino acids such as arginine and lysine, amine derivatives and salts, amino sugars such as meglumine, salts of stearic acid such as sodium stearate and the like, with each possibility representing a separate embodiment. Suitable inorganic alkaline agents include, but are not limited to, hydroxides such as sodium or potassium hydroxide, carbonates such as calcium, magnesium or zinc carbonate and the like, with each possibility representing a separate embodiment. In particular embodiments, the orally disintegrating composition does not contain antacids. In some embodiments, the alkalizing agent in the cores is in an amount of about 0.2% to about 10% by weight of the total mass of orally disintegrating composition, including each integer within the specified range. In one embodiment, the alkalizing agent in the cores is in an amount of about 0.3% to about 8% by weight of the total mass of the orally disintegrating composition, including each integer within the specified range. In another embodiment, the alkalizing agent in the cores is in an amount of about 1% to about 5% by weight of the total mass of the orally disintegrating composition, including each integer within the specified range. In certain embodiments, the alkalizing agent in the cores is in an amount of about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the total mass of the orally disintegrating composition, with each possibility representing a separate embodiment. In one embodiment, the alkalizing agent in the cores comprises meglumine. In another embodiment, the alkalizing agent in the cores comprises sodium stearate.

In some embodiments, the cores (either active cores or inert seeds coated with a drug layer comprising a PPI) are in an amount of about 5% to about 50% of the total orally disintegrating composition mass, including each integer within the specified range. In other embodiments, the cores are in an amount of about 5% to about 40% of the total orally disintegrating composition mass, including each integer within the specified range. In certain embodiments, the plurality of cores are in an amount of about 5%, about 10%, about 15%, about 20%, about 25%, about 30%, about 35%, about 40%, about 45%, or about 50% of the total orally disintegrating composition mass, with each possibility representing a separate embodiment.

According to the principles provided herein, the cores are further coated with coating layer(s) including an optional subcoating, an enteric coating and/or an optional taste-masking coating.

In some embodiments, the cores are coated with a subcoating layer designed to minimize or substantially prevent an interaction between the enteric coating layer having free carboxyl groups and the alkaline core that comprises one or more PPIs which are often acid-labile. The subcoating layer typically provides physical separation between the alkaline core containing one or more PPIs and the acidic enteric coating. In certain embodiments, the subcoating layer comprises one or more of a binder, a filler, and a surfactant as described hereinabove, with each possibility representing a separate embodiment. In further embodiments, the subcoating layer may further comprise an anti-tacking agent.

In some embodiments, the subcoating layer comprises a binder in an amount of about 0.5% to about 15% of the total orally disintegrating composition mass, including each integer within the specified range. In other embodiments, the binder in the subcoating layer is in an amount of about 0.5% to about 10% of the total orally disintegrating composition mass, including each integer within the specified range. In certain embodiments, the binder in the subcoating layer is in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% of the total orally disintegrating composition mass, with each possibility representing a separate embodiment. In one embodiment, the binder in the subcoating layer comprises hydroxypropylmethyl cellulose. In other embodiments, the binder in the subcoating layer comprises povidone or copovidone.

In some embodiments, the subcoating layer comprises a filler in an amount of about 0.5% to about 15% of the total orally disintegrating composition mass, including each integer within the specified range. In one embodiment, the filler in the subcoating layer is in an amount of about 0.5% to about 10% of the total orally disintegrating composition mass, including each integer within the specified range. In yet another embodiment, the filler in the subcoating layer is in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% of the total orally disintegrating composition mass, with each possibility representing a separate embodiment. In some embodiments, the filler in the subcoating layer comprises mannitol.

In various embodiments, the subcoating layer comprises a surfactant in an amount of about 0.01% to about 8% by weight of the total mass of the orally disintegrating composition, including each integer within the specified range. In several embodiments, the surfactant in the subcoating layer is in an amount of about 0.01% to about 5% by weight of the total mass of the orally disintegrating composition, including each integer within the specified range. In additional embodiments, the surfactant in the subcoating layer is in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, or about 8% by weight of total mass of the orally disintegrating composition, with each possibility representing a separate embodiment. In certain embodiments, the surfactant in the subcoating layer comprises polysorbate.

In some embodiments, the subcoating layer comprises an anti-tacking agent. Suitable anti-tacking agents include, but are not limited to, magnesium stearate, calcium stearate, stearic acid, talc, colloidal silicon and the like among others. Each possibility represents a separate embodiment. In various embodiments, the anti-tacking agent in the subcoating layer is in an amount of about 0.01% to about 10% of the total orally disintegrating composition mass, including each integer within the specified range. In other embodiments, the anti-tacking agent in the subcoating layer is in an amount of about 0.01% to about 8% of the total orally disintegrating composition mass, including each integer within the specified range. In further embodiments, the anti-tacking agent in the subcoating layer is in an amount of about 0.01%, about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% of the total orally disintegrating composition mass, with each possibility representing a separate embodiment. In certain embodiments, the anti-tacking agent in the subcoating layer comprises talc.

In some embodiments, the subcoating layer (if present) is in an amount of about 2% to about 30% of the total orally disintegrating composition mass, including each integer within the specified range. In other embodiments, the subcoating layer is in an amount of about 2% to about 20% of the total orally disintegrating composition mass, including each integer within the specified range. In yet other embodiments, the subcoating layer is in an amount of about 5% to about 15% of the total orally disintegrating composition mass, including each integer within the specified range. In certain embodiments, the subcoating layer is in an amount of about 2%, about 4%, about 6%, about 8%, about 10%, about 12%, about 14%, about 16%, about 18%, about 20%, about 22%, about 24%, about 26%, about 28%, or about 30% of the total orally disintegrating composition mass, with each possibility representing a separate embodiment.

In some embodiments, the first population of particles comprises an enteric coating, which protects the PPI from the acidic environment of the stomach. The enteric coating includes one or more enteric polymers and optionally other pharmaceutically acceptable excipients, such as a plasticizer, a glidant, and colorant with each possibility representing a separate embodiment. In additional embodiments, the enteric coating may further comprise an anti-tacking agent as described hereinabove. In some embodiments, the enteric coating is applied directly to the cores comprising the PPI. In other embodiments, the enteric coating is applied to the subcoating layer, which is overcoating the cores. Generally, enteric coatings include pH dependent polymers. These polymers are typically characterized by increase in permeability at pH values of above pH 5.0 (e.g., intestinal fluid) while remaining insoluble at low pH values, such as those found in the environment of the stomach.

Exemplary and non-limiting enteric polymers include hydroxypropyl methylcellulose phthalate (HPMCP), hydroxypropyl methylcellulose acetate succinate (HPMCAS), acrylic and methacrylate acid copolymers, cellulose acetate phthalate (CAP), cellulose acetate butyrate, polyvinyl acetate phthalate, cellulose acetate trimellitate, alginic acid salts, such as sodium or potassium alginate, and shellac. Each possibility represents a separate embodiment. Acrylic and methacrylate acid copolymers are anionic copolymers based on (meth)acrylic acid and alkyl (meth)acrylate, such as, but not limited to, polymethacrylic acid, polymethyl methacrylate, polyethyl methacrylate, and polyethyl acrylate among others. Commercial acrylic acid and methacrylate acid copolymers are available under the trade name Eudragit® (Evonik Industries AG, Essen, Germany) and are typically provided as powder or aqueous dispersions, including, but not limited to, Eudragit® L 30 D-55; Eudragit® L 100-55; Eudragit® L 100; Eudragit® L 12.5; Eudragit® NE 40 D, Eudragit® RL 100, Eudragit® S 100; Eudragit® S 12.5; Eudragit® FS 30 D; Eudragit® RL PO; Eudragit® RL 12.5, Eudragit® RL 30 D; Eudragit® RS 100; Eudragit® RS PO; Eudragit® RS 30 D; Eudragit® RS 12.5; Eudragit® NE 30 D; Eudragit® NM 30 D; or combinations and mixtures thereof. In certain embodiments, the enteric polymer in the enteric coating comprises hydroxypropyl methylcellulose phthalate (HPMCP). In other embodiments, the enteric polymer in the enteric coating comprises hydroxypropyl methylcellulose acetate succinate (HPMCAS).

In some embodiments, the enteric polymer is in an amount of about 5% to about 30% of the total orally disintegrating composition mass, including each integer within the specified range. In other embodiments, the enteric polymer is in an amount of about 5% to about 25% of the total orally disintegrating composition mass, including each integer within the specified range. In certain embodiments, the enteric polymer is in an amount of about 5%, about 6%, about 7%, about 8%, about 9%, about 10%, about 12%, about 14%, about 16%, about 18%, about 20%, about 22%, about 24%, about 26%, about 28%, or about 30% of the total orally disintegrating composition mass, with each possibility representing a separate embodiment.

In some embodiments, the enteric coating further comprises one or more plasticizers. Plasticizers are known to increase the flexibility of the coating and help minimize or substantially prevent cracking of the enteric coat upon compression. Further, plasticizers may also increase the adhesion of the enteric coating polymer chains. Suitable plasticizers include, but are not limited to, cetyl alcohol, dibutyl sebacate, polyethylene glycol, polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol among others. Each possibility represents a separate embodiment. In some embodiments, the one or more plasticizers in the enteric coating are in an amount of about 0.1% to about 15% of the total orally disintegrating composition mass, including each integer within the specified range. In other embodiments, the one or more plasticizers in the enteric coating are in an amount of about 0.1% to about 10% of the total orally disintegrating composition mass, including each integer within the specified range. In certain embodiments, the one or more plasticizers in the enteric coating are in an amount of about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, or about 15% of the total orally disintegrating composition mass, with each possibility representing a separate embodiment. In certain embodiments, the one or more plasticizers in the enteric coating comprise triethyl citrate, cetyl alcohol, and a mixture or combination thereof.

In various embodiments, the enteric coating further comprises a glidant. A suitable glidant within the scope of the present disclosure is e.g., colloidal silicon dioxide. Typically the glidant in the enteric coating is in an amount of about 0% to about 5% of the total orally disintegrating composition mass, including each integer within the specified range. In certain embodiments, the glidant in the enteric coating is in an amount of about 0%, about 0.5%, about 1%, about 1.5%, about 2%, about 2.5%, about 3%, about 3.5%, about 4%, about 4.5%, or about 5% of the total orally disintegrating composition mass, with each possibility representing a separate embodiment.

In other embodiments, the enteric coating further comprises a colorant. Suitable colorants include, but are not limited to, alumina (dried aluminum hydroxide), annatto extract, calcium carbonate, canthaxanthin, caramel, 0-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide. Each possibility represents a separate embodiment. In certain embodiments, the colorant in the enteric coating is in an amount of about 0% to about 5% of the total orally disintegrating composition mass, including each integer within the specified range. In other embodiments, the colorant in the enteric coating is in an amount of about 0%, about 0.5%, about 1%, about 1.5%, about 2%, about 2.5%, about 3%, about 3.5%, about 4%, about 4.5%, or about 5% of the total orally disintegrating composition mass, with each possibility representing a separate embodiment. In certain embodiments, the colorant in the enteric coating comprises titanium dioxide.

In various embodiments, the enteric coating further comprises an anti-tacking agent. In some embodiments, the anti-tacking agent in the enteric coating is in an amount of about 0% to about 15% of the total orally disintegrating composition mass, including each integer within the specified range. In further embodiments, the anti-tacking agent in the enteric coating is in an amount of about 0.1% to about 10% of the total orally disintegrating composition mass, including each integer within the specified range. In other embodiments, the anti-tacking agent in the enteric coating is in an amount of about 0%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% of the total orally disintegrating composition mass, with each possibility representing a separate embodiment. In certain embodiments, the anti-tacking agent in the enteric coating comprises talc.

In some embodiments, the weight of the enteric coating is about 5% to about 50% of the total orally disintegrating composition mass, including each integer within the specified range. In one embodiment, the weight of the enteric coating is about 10% to about 30% of the total orally disintegrating composition mass, including each integer within the specified range. In another embodiment, the weight of the enteric coating is about 10% to about 25% of the total orally disintegrating composition mass, including each integer within the specified range. In other embodiments, the weight of the enteric coating is about 5%, about 10%, about 15%, about 20%, about 25%, about 30%, about 35%, about 40%, about 45%, or about 50% by weight of the total orally disintegrating composition mass, with each possibility representing a separate embodiment.

In certain embodiments, the enteric coating in the first population of particles is the outermost coating layer in said population. In accordance with these embodiments, the first population of particles is devoid of a coating layer, e.g., a deformable layer, over the enteric coating. In some embodiments, the first population of particles comprising an enteric overcoating is devoid of any barrier separating the enteric coated cores of the first population from the particles of the second population. In alternative embodiments, the first population of particles comprises a taste-masking coating over the enteric coating whereby the taste-masking coating reduces the taste sensation of PPIs characterized by bitter or unpleasant taste. The taste-masking coating typically comprises polymers which are practically insoluble at pH values greater than those found in the stomach i.e. at pH values greater than 5.0 while being soluble at acidic pH values. In some embodiments, the polymer in the taste-masking coating comprises a (meth)acrylate polymer or copolymer, such as acrylate and methacrylate copolymers having primary, secondary or tertiary amino groups or quaternary ammonium groups. Commercially available taste-masking polymers include, but are not limited to, Eudragit® E 100; Eudragit® E 12.5; Eudragit® EPO; or Eudragit® RL 100 (Evonik Industries). Each possibility represents a separate embodiment.

In some embodiments, the polymer in the taste-masking coating is in an amount of about 0.5% to about 15% of the total orally disintegrating composition mass, including each integer within the specified range. In other embodiments, the polymer in the taste-masking coating is in an amount of about 0.5% to about 10% of the total orally disintegrating composition mass, including each integer within the specified range. In certain embodiments, the polymer in the taste-masking coating is in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% of the total orally disintegrating composition mass, with each possibility representing a separate embodiment. In one embodiment, the polymer in the taste-masking coating comprises a methyl methacrylate-butyl methacrylate-dimethylaminoethyl methacrylate copolymer (e.g., poly(butyl methacrylate-co-(2-dimethylaminoethyl) methacrylate-co-methyl methacrylate) 1:2:1).

In some embodiments, the taste-masking coating further comprises one or more pharmaceutically acceptable excipients, such as a glidant or colorant described hereinabove. In one embodiment, the glidant in the taste-masking coating is in an amount of about 0% to about 5% of the total orally disintegrating composition mass, including each integer within the specified range. In certain embodiments, the glidant in the taste-masking coating is in an amount of about 0%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% of the total orally disintegrating composition mass, with each possibility representing a separate embodiment. In one embodiment, the glidant in the taste-masking coating comprises colloidal silicon dioxide. In one embodiment, the colorant in the taste-masking coating is in an amount of about 0% to about 5% of the total orally disintegrating composition mass, including each integer within the specified range. In certain embodiments, the colorant in the taste-masking coating is in an amount of about 0%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, or about 5% of the total orally disintegrating composition mass, with each possibility representing a separate embodiment. In one embodiment, the colorant in the taste-masking coating comprises ferric oxide.

In certain embodiments, the weight of the taste-masking coating (if present) is about 0.5% to about 25% of the total orally disintegrating composition mass, including each integer within the specified range. In other embodiments, the weight of the taste-masking coating is about 1% to about 15% by weight of the total orally disintegrating composition mass, including each integer within the specified range. In yet other embodiments, the weight of the taste-masking coating is about 0.5%, about 1%, about 2%, about 4%, about 6%, about 8%, about 10%, about 15%, about 20%, or about 25% by weight of the total orally disintegrating composition mass, with each possibility representing a separate embodiment.

In some embodiments, the coating layer(s) described herein substantially cover the cores or the inner layer onto which they are applied. In other embodiments, the coating layer(s) cover the cores or the inner layer onto which they are applied by at least about 25% of the surface area. In particular embodiment, the coating layer(s) cover the cores or the inner layer onto which they are applied by at least about 25%, about 30%, about 35%, about 40%, about 45%, about 50%, about 55%, about 60%, about 65%, about 70%, about 75%, about 80%, about 85%, about 90%, about 95% or about 100% (substantially complete coverage) of the surface area, with each possibility representing a separate embodiment.

In various embodiments, the first population of particles has a crush strength of about 300 g or less. In one embodiment, the first population of particles has a crush strength of about 100 g to about 300 g, including each integer within the specified range. In another embodiment, the first population of particles has a crush strength of about 50 g, about 75 g, about 100 g, about 125 g, about 150 g, about 175 g, about 200 g, about 225 g, about 250 g, about 275 g, or about 300 g, with each possibility representing a separate embodiment.

In certain aspects and embodiments, the first population of particles comprises the composition detailed in Table 1.

TABLE 1

| | | |
|---|---|---|
| | | Wt % of the |
| Material Class | Exemplary Material(s) | total composition |

Cores

| | | |
|---|---|---|
| PPI | Lansoprazole | 1-15 |
| Inert seeds | Sugar spheres or micro- | 2-20 |
| | crystalline cellulose | |
| | (MCC) particles | |
| Binder | Hydroxypropylmethyl | 0.01-8 |
| | cellulose (HPMC) or | |
| | polyvinylpyrrolidone | |
| | (PVP) | |
| Filler (optional) | Mannitol | 0-10 |
| Surfactant (optional) | Polysorbate | 0-8 |
| Alkalizing agent | Meglumine or sodium stearate | 0.2-10 |

| | |
|---|---|
| Wt % of the total orally | 5-50 |
| disintegrating composition mass | |

Subcoating Layer (optional)

| | | |
|---|---|---|
| Binder | HPMC or PVP | 0.5-15 |
| Filler | Mannitol | 0.5-15 |
| Surfactant | Polysorbate | 0.01-8 |
| Anti-tacking agent | Talc | 0.01-10 |

| | |
|---|---|
| Wt % of the total orally | 2-30 |
| disintegrating composition mass | |

Enteric Coating

| | | |
|---|---|---|
| Enteric Polymer | HPMC phthalate or HPMC | 5-30 |
| | acetate succinate | |
| Plasticizer | Triethyl citrate or | 0.1-15 |
| | cetyl alcohol | |
| Anti-tacking agent | Talc | 0-15 |
| (optional) | | |
| Colorant (optional) | Titanium dioxide | 0-5 |
| Glidant (optional) | Colloidal silicon dioxide | 0-5 |

| | |
|---|---|
| Wt % of the total orally | 5-50 |
| disintegrating composition mass | |

Taste-masking coating (optional)

| | | |
|---|---|---|
| Taste-masking | Amino methacrylate | 0.5-15 |
| polymer | copolymer | |
| Glidant (optional) | Colloidal silicon dioxide | 0-5 |
| Colorant (optional) | Ferric oxide | 0-5 |

| | |
|---|---|
| Wt % of the total orally | 0.5-25 |
| disintegrating composition mass | |

2^{nd} Population of Particles

In some embodiments, the orally disintegrating composition according to the principles described herein comprises a second population of particles comprising a disintegrant.

According to certain aspects and embodiments, the second population of particles has a median particle size in the range of about 50 μm to about 250 μm, including each integer within the specified range. In some embodiments, the second population of particles has a median particle size in the range of about 100 μm to about 200 μm, including each integer within the specified range. In various embodiments, the second population of particles has a median particle size of about 50 μm, about 60 μm, about 70 μm, about 80 μm, about 90 μm, about 100 μm, about 110 μm, about 120 μm, about 130 μm, about 140 μm, 150 μm, about 160 μm, about 170 μm, about 180 μm, about 190 μm, 200 μm, about 210 μm, about 220 μm, about 230 μm, about 240 μm, or about 250 μm, with each possibility representing a separate embodiment. In further embodiments, the second population of particles has a d₁₀ particle size in the range of about 1 μm to about 100 μm, including each integer within the specified range. In additional embodiments, the second population of particles has a d₁₀ particle size in the range of about 5 μm to about 50 μm, including each integer within the specified range. In various embodiments, the second population of particles has a d₉₀ particle size in the range of about 250 μm to about 500 μm, including each integer within the specified range. In other embodiments, the second population of particles has a d₉₀ particle size in the range of about 300 μm to about 450 μm, including each integer within the specified range.

In certain embodiments, the second population of particles (also termed powder mixture) comprises a disintegrant and optionally one or more pharmaceutically acceptable excipients such as, but not limited to, a binder, a filler, a surfactant, a glidant, a plasticizer, an anti-tacking agent, an alkalizing agent, and a colorant as described hereinabove. Each possibility represents a separate embodiment. Optionally, the second population of particles further comprises at least one of a diluent, a lubricant, a tonicity enhancing agent, a wetting agent, a buffering substance, a preservative, a flavoring agent, an anti-oxidant or a mixture or combination thereof. Each possibility represents a separate embodiment.

Suitable disintegrants within the scope of the present disclosure include, but are not limited to, crospovidone, croscarmellose sodium, a sugar alcohol, a cellulose derivative, cross-linked derivatives of starch (e.g. sodium starch glycolate), pregelatinized starch, cross-linked sodium carboxymethyl cellulose, low substituted hydroxypropylcellulose and any combination or mixture thereof, with each possibility representing a separate embodiment. Additional disintegrants include, but are not limited to, silicates, carbonates, polyoxyethylene sorbitan fatty acid esters, stearic monoglyceride, guar gum, and lactose. Each possibility represents a separate embodiment. Suitable sugar alcohols include, but are not limited to, mannitol, sorbitol, maltitol, xylitol, and any combination or mixtures thereof. Each possibility represents a separate embodiment. Additional sugar alcohols include, but are not limited to, arabitol, isomalt, erythritol, glycerol, lactitol, and any combination or mixtures thereof. Each possibility represents a separate embodiment. Suitable cellulose derivatives include, but are not limited to, methylcellulose, cross-linked carboxymethyl celluloses, microcrystalline cellulose and any combination or mixtures thereof. Each possibility represents a separate embodiment.

In some embodiments, the disintegrant in the second population of particles is in an amount of about 1% to about 25% of the total orally disintegrating composition mass, including each integer within the specified range. In other embodiments, the disintegrant in the second population of particles is in an amount of about 2% to about 20% of the total orally disintegrating composition mass, including each integer within the specified range. In yet other embodiments, the disintegrant in the second population of particles is in an amount of about 2% to about 15% of the total orally disintegrating composition mass, including each integer within the specified range. In certain embodiments the disintegrant in the second population of particles is in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% of the total orally disintegrating composition mass, with each possibility representing a separate embodiment. In one embodiment, the disintegrant in the second population of particles comprises crospovidone.

Suitable diluents include, but are not limited to, dicalcium phosphate dihydrate, sugars, lactose, calcium phosphate, cellulose, kaolin, mannitol, sodium chloride, and dry starch. Each possibility represents a separate embodiment.

Suitable lubricants include, but are not limited to, sodium stearyl fumarate, stearic acid, polyethylene glycol or stearates, such as magnesium stearate. Each possibility represents a separate embodiment.

Suitable tonicity enhancing agents are selected from ionic and non-ionic compounds. For example, ionic compounds include, but are not limited to, alkali metal or alkaline earth metal halides, such as, for example, CaCl₂ KBr, KCl, LiCl, NaI, NaBr or NaCl, or boric acid. Non-ionic tonicity enhancing agents are, for example, urea, glycerol, sorbitol, mannitol, propylene glycol, or dextrose. Each possibility represents a separate embodiment.

Suitable wetting agents include, but are not limited to, glycerin, starches, and the like. Each possibility represents a separate embodiment.

Suitable buffering substances include, but are not limited to, acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide and magnesium hydroxide. Each possibility represents a separate embodiment.

Examples of preservatives are quaternary ammonium salts such as benzalkonium chloride, benzoxonium chloride or polymeric quaternary ammonium salts, alkyl-mercury salts of thiosalicylic acid, such as, for example, thiomersal, phenylmercuric nitrate, phenylmercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sorbic acid or ascorbic acid. Each possibility represents a separate embodiment.

Suitable flavoring agents include, but are not limited to, sweeteners such as sucralose and synthetic flavor oils and flavoring aromatics, natural oils, extracts from plants, leaves, flowers, and fruits, and combinations thereof. Exemplary flavoring agents include cinnamon oils, oil of wintergreen, peppermint oils, clover oil, hay oil, anise oil, eucalyptus, vanilla, citrus oil such as lemon oil, orange oil, grape and grapefruit oil, and fruit essences including apple, peach, pear, strawberry, raspberry, cherry, plum, pineapple, and apricot. Each possibility represents a separate embodiment.

Exemplary and non-limiting anti-oxidants include tocopherols (e.g., alpha-tocopherol, beta-tocopherol, gamma-tocopherol, or delta-tocopherol), butylated hydroxytoluene (BHT), butylated hydroxyanisole (BHA), citric acid, ascorbic acid, phenolic diterpenes (e.g., carnosic acid, carnosol, rosmanol, epirosmanol, isorosmanol, or methyl carnosate), rosmarinic acid, eugenol, eugenyl acetate, clove bud extract, methanolic extract, tea catechins (e.g., epigallocatechin gallate, epicatechin gallate, epigallocatechin, or epicatechin), or a mixture or combination thereof. Each possibility represents a separate embodiment.

In certain aspects and embodiments, the second population of particles comprises the composition detailed in Table 2.

TABLE 2

| | | Wt % of the |
|---|---|---|
| Material Class | Exemplary Material(s) | total composition |
| Disintegrant | Crospovidone or sugar alcohol | 1-25 |
| Filler | MCC or mannitol | 5-50 |
| Binder | HPMC or PVP | 0-35 |
| Flavoring agent | Sucralose or strawberry flavor | 0-5 |
| Antioxidant | Ascorbic acid | 0-5 |
| Glidant | Colloidal silicon dioxide | 0-5 |
| Lubricant | Sodium stearyl fumarate | 0-5 |
| Anti-tacking agent | Talc | 0-5 |
| Colorant | Ferric oxide | 0-5 |

Ratio Between 1^{st} and 2^{nd} Populations:

In some embodiments, the weight percent ratio of the first population of particles (i.e., the cores comprising a PPI including the coating layer(s) described herein) to the second population of particles (i.e., the powder mixture) is about 0.1:1 to about 1:0.1, including all iterations of ratios within the specified range. In other embodiments, the weight percent ratio of the first population of particles to the second population of particles is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1, with each possibility representing a separate embodiment. In one embodiment, the weight percent ratio of the first population of particles to the second population of particles is about 0.5:1. In another embodiment, the weight percent ratio of the first population of particles to the second population of particles is about 1:1. In further embodiments, the weight percent ratio of the first population of particles to the second population of particles is about 1:0.8. In additional embodiments, the weight percent ratio of the first population of particles to the second population of particles is about 1:0.5.

In some embodiments, the orally disintegrating composition described herein is an orally disintegrating tablet comprising:
A first population of particles comprising:
   inert seeds comprising a filler, e.g. sugar spheres and/or microcrystalline cellulose particles;
   a drug coating layer over the inert seeds, the drug coating layer comprising a PPI, e.g. lansoprazole; an alkalizing agent, e.g. meglumine; a binder, e.g. HPMC and/or PVP; a surfactant, e.g. polysorbate; and/or a filler, e.g. mannitol;
   a subcoating layer over the drug coating layer, the subcoating layer comprising a binder, e.g. HPMC and/or PVP; an anti-tacking agent, e.g. talc; a surfactant, e.g. polysorbate and/or a filler, e.g. mannitol;
   an enteric coating over the subcoating layer, the enteric coating comprising an enteric polymer, e.g. HPMC phthalate and/or HPMC acetate succinate; an anti-tacking agent, e.g. talc; a plasticizer, e.g. cetyl alcohol and/or triethyl citrate; and/or a colorant, e.g. titanium dioxide;
A second population of particles comprising one or more disintegrants, e.g. crospovidone and/or sugar alcohol; and a filler, e.g. MCC and/or mannitol; a binder, e.g. HPMC and/or PVP; a flavoring agent, e.g. sucralose and/or strawberry flavor; an anti-oxidant, e.g. ascorbic acid; a glidant, e.g. colloidal silicon dioxide; a lubricant, e.g. sodium stearyl fumarate; and/or a colorant e.g. ferric oxide.

The orally disintegrating compositions disclosed herein can be manufactured using conventional methods as is known in the art. According to certain embodiments, the method comprises preparing a solution or dispersion comprising a PPI, a subcoating solution or dispersion, an enteric coating solution or dispersion, and/or a taste-masking solution or dispersion. In various embodiments, suitable solvents are used to dissolve or suspend one or more of the coating mixture ingredients. Such solvents include, but are not limited to, water, protic or aprotic organic solvents. Exemplary and non-limiting protic or aprotic organic solvents include isopropyl alcohol, ethanol, and acetone or a mixture or combination thereof, with each possibility representing a separate embodiment.

The prepared solutions or dispersions can be applied to the seeds or cores to generate the first population of particles described herein by conventional coating techniques known in the art, see, Remington, J. P.; Beringer, P. Remington: The Science and Practice of Pharmacy; Lippincott Williams & Wilkins: Philadelphia, 2006. For example, fluidized coating methods including, but not limited to, pan coating, rotary bed coater, Wurster fluidized bed coating, fluidized bed bottom sprayed coating or a turbo jet-technology can be used. A fluidized bed is a bed of solid particles which are suspended in a stream of air or gas passing upward through the particles, in which the coating material is aerosolized. As the air travels through the particle bed, the particles are mixed in the stream of gas or air with the coating material, thereby being coated and also dried.

Additional coating techniques within the scope of the disclosure include extruder or spray dryer. When using spray coating technique, various apparatus may be employed including, but not limited to, rotary disks, single-fluid high pressure swirl nozzles, two-fluid nozzles or ultrasonic nozzles, single stage dryer, two stage dryer, horizontal dryer, multi stage drier, compact spray dryer, integrated filter drier, Filtermat® dryer, including, e.g., Glatt, Gea-Niro, BWI Hüttlin, and Allgaier among others, with each possibility representing a separate embodiment.

Alternatively, a dry powder layering may be used to apply the coating layers. Dry powder coating processes may be performed using known systems, such as, for example, CF-Granulator (Freund Industrial, Tokyo, Japan), Granurex (Vector Corporation, Marion, Iowa, USA), GS HP/25 equipment (GS Coating System, Italy), Centrifugal Fluid Bed Granulator (Glatt, Germany) and other appropriate systems known in the art.

At the end of the each step of coating, a step of drying may be applied to allow any residual solvent to evaporate. The rate, amount, homogeneity, inter- and intra-uniformity, efficiency, quality, and yield of the coating may be controlled by parameters such as batch size, rotor speed, binder spray rate, powder addition rate, inlet and outlet air temperature, bed temperature, atomization air pressure, air flap and air flow as is known in the art.

In some embodiments, the method of manufacturing an orally disintegrating composition according to the disclosure comprises: (a) generating a plurality of cores comprising a therapeutically effective amount of a PPI; (b) applying a solution or dispersion comprising an enteric polymer to the plurality of cores of step (a) thereby obtaining a plurality of enteric coated cores constituting the first population of particles; and (c) mixing the first population of particles with a second population of particles comprising a disintegrant. In further embodiments, the method further comprises (d) compressing the mixture of step (c) thereby obtaining an orally disintegrating tablet. In certain embodiments, the step (a) of generating the plurality of cores comprises applying a solution or dispersion comprising a therapeutically effective amount of a PPI to a plurality of inert seeds. In other embodiments, the method for manufacturing further comprises an additional step prior to step (b) of applying the enteric coating, the additional step (al) comprising: applying a subcoating solution or dispersion comprising at least one of hydroxypropyl methylcellulose, hydroxypropyl cellulose, polyvinylpyrrolidone, polyethylene glycol, polyvinyl alcohol or a mixture or combination thereof to the plurality of cores of step (a) thereby obtaining a subcoating between the cores and the enteric coating. In additional embodiments, the method of manufacturing further comprises an additional step prior to step (c) of mixing the first and second populations of particles, the additional step (b1) comprising: applying a taste-masking solution or dispersion comprising at least one taste-masking polymer to the plurality of enteric coating cores of step (b) thereby obtaining a taste-masking coating over the enteric coating.

In some embodiments, the methods of manufacturing further comprise the steps of sieving the coated cores between each coating step. It is believed that the sieving steps eliminate oversized agglomerates. Optionally, the methods of manufacturing further comprise additional processing steps including, but not limited to heating, drying, lubricating, and packaging as is known in the art.

The steps of mixing the first and second populations as well as the preparation of the powder mixture constituting the second population of particles can be performed using any pharmaceutical blending process known in the art. For example, they can be achieved using any suitable type of mixer or blender. Non-limiting examples include simple paddle mixer, ribbon and/or tumbling mixers, plow blenders and drum agglomerators, V-blenders, double cone blenders, slant cone blenders, twin shell blenders, e.g., Patterson Kelley V Blenders, Gemco double cone blenders, diffusion blenders and the like.

In certain embodiments, the composition is compressed to an orally disintegrating tablet. The compression process may be achieved using any suitable tableting equipment. Non-limiting examples include mini press, single or double punch or rotary tablet press such as Killian, Korsch, Colton, Manesty, Stokes, Vector and the like, among others.

In some embodiments, the orally disintegrating tablet has a hardness of about 20 N to about 100 N, including each integer within the specified range. In other embodiments, the orally disintegrating tablet has a hardness of about 20 N to about 80 N, including each integer within the specified range. In yet other embodiments, the orally disintegrating tablet has a hardness of about 30 N to about 70 N, including each integer within the specified range. In additional embodiments, the orally disintegrating tablet has a hardness of about 20 N, about 25 N, about 30 N, about 35 N, about 40 N, about 45 N, about 50 N, about 55 N, about 60 N, about 65 N, about 70 N, about 75 N, about 80 N, about 85 N, about 90 N, about 95 N, or about 100 N, with each possibility representing a separate embodiment.

In certain embodiments, the orally disintegrating tablet has friability of about 1% or less. In other embodiments, the orally disintegrating tablet has friability of about 1%, about 0.95%, about 0.9%, about 0.85%, about 0.8%, about 0.75%, about 0.7%, about 0.65%, about 0.6%, about 0.55% about 0.5%, about 0.45%, about 0.4%, about 0.35%, about 0.3%, about 0.25%, about 0.2%, about 0.15%, about 0.1%, about 0.05%, or about 0%, with each possibility representing a separate embodiment.

The orally disintegrating pharmaceutical composition disclosed herein is useful in inhibiting gastric acid secretion. In certain embodiments, the orally disintegrating pharmaceutical composition is useful in the treatment of a disease or disorder selected from the group consisting of gastroesophageal reflux disease, gastritis, peptic ulcers (duodenal and gastric) and erosive esophagitis, with each possibility representing a separate embodiment.

Accordingly, there is provided a method of inhibiting gastric acid secretion in the treatment of a disease or disorder selected from the group consisting of gastroesophageal reflux disease, gastritis, peptic ulcers (duodenal and gastric) and erosive esophagitis, the method comprising administering to a subject in need thereof the orally disintegrating composition disclosed herein. In some embodiments, the method further comprises the disintegration of the composition in the oral cavity of the subject to provide a plurality of particles as described herein, the method further comprises the release of a majority of the PPI from the plurality of particles in the upper intestine.

The subject in need thereof is typically a mammal, preferably a human. The orally disintegrating composition may be administered in a solid dosage form to be placed on the tongue (lingual administration), or under the tongue (sublingual administration), or applied to the buccal mucosa (buccal administration). Lingual administration typically stimulates saliva generation, which enhances disintegration of the composition. In some embodiments, the composition is a dosage form suitable for forming a suspension of undissolved particles in saliva, which can then be swallowed, allowing for absorption of the PPI in the GI tract, generally in the upper intestine. The amount of a composition to be administered depends on various factors including the subject being treated (age and gender) and the severity of the disease, and can be determined by the judgment of the prescribing physician. Because of patient-to-patient variability, dosages are a guideline only and the physician may adjust doses of the compounds to achieve the level of effective treatment that the physician considers appropriate for the patient. In considering the degree of treatment desired, the physician must balance a variety of factors such as the age of the patient and the presence of other diseases or conditions. The orally disintegrating compositions may contain any dosage of the PPI, for example from about 2 mg to about 50 mg of the active ingredient such as, but not limited to, 5 mg, 10 mg, 15 mg, 20 mg, 25 mg, 30 mg, 35 mg, 40 mg, 45 mg, or 50 mg, with each possibility representing a separate embodiment.

The term “therapeutically effective amount” or “an effective amount” as used herein refers to a quantity of a compound which is sufficient to provide a beneficial effect to the subject to which the compound is administered. The effective amount, according to the principles disclosed herein can be determined by any one of ordinary skill in the art and can be tested on various models both in vitro and in vivo.

The term “treating” as used herein refers to stopping or slowing down the progression of the disease. The term “treating” further includes the reduction in the occurrence of various symptoms associated with gastric acid secretion.

As used herein and in the appended claims, the term “about” refers to ±10%.

As used herein and in the appended claims, the singular forms “a”, “an”, and “the” include plural references unless the context clearly dictates otherwise. Thus, for example, reference to “a layer” includes a plurality of such layers and equivalents thereof known to those skilled in the art, and so forth. It should be noted that the term “and” or the term “or” are generally employed in its sense including “and/or” unless the context clearly dictates otherwise.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

An orally disintegrating composition according to certain embodiments of the disclosure was prepared as follows: A first population of particles was prepared by coating inert sugar spheres with a drug layer containing 15 mg lansoprazole and typically further containing a binder (e.g. HPMC) and an alkalizing agent (e.g. meglumine) to obtain cores. A subcoating layer containing HPLC and typically an anti-tacking agent (e.g. talc), and an enteric coating layer containing enteric polymer such as HPMC phthalate or HPMC acetate succinate and typically further containing a plasticizer (e.g. triethyl citrate) and an anti-tacking agent (e.g. talc) were then sequentially applied to the cores. A second population of particles containing a disintegrant (e.g. crospovidone, Pharmaburst® (see p. 13 of WO 2006/058250)) was prepared by mixing the disintegrant with excipient(s) typically including a glidant (e.g. colloidal silicon dioxide), and a lubricant (e.g. sodium stearyl fumarate).

Exemplary compositions according to certain embodiments of the disclosure are outlined in Tables 3-4 below:

TABLE 3

| | |
|---|---|
| Materials | Amount (mg) |

1^{st} population of particles

| | |
|---|---|
| Lansoprazole | 15.0 |
| Sugar spheres | 21.0 |
| Mannitol | 16.5 |
| Meglumine | 4.6 |
| Polysorbate | 4.7 |
| HPMC | 18.3 |
| Talc | 5.1 |
| HPMC phthalate | 30.2 |
| cetyl alcohol | 5.5 |
| Triethyl citrate | 2.5 |
| Titanium dioxide | 0.6 |

2^{nd} population of particles

| | |
|---|---|
| Pharmaburst ® | 108.5 |
| Crospovidone | 8.3 |
| Sucralose | 0.5 |
| Ascorbic acid | 2.4 |
| Strawberry flavor | 1.2 |
| Colloidal silicon dioxide | 1.3 |
| Sodium stearyl fumarate | 1.9 |

TABLE 4

| | |
|---|---|
| Materials | Amount (mg) |

1^{st} population of particles

| | |
|---|---|
| Lansoprazole | 15.0 |
| Sugar spheres | 21.0 |
| Mannitol | 13.8 |
| Meglumine | 4.6 |
| Polysorbate | 4.2 |
| HPMC | 14.4 |
| Talc | 14.1 |
| HPMC acetate succinate | 43.0 |
| Titanium dioxide | 0.5 |
| Triethyl citrate | 6.4 |
| Colloidal silicon dioxide | 1.0 |

2^{nd} population of particles

| | |
|---|---|
| Pharmaburst ® | 92.0 |
| Crospovidone | 12.8 |
| Sucralose | 0.5 |
| Ascorbic acid | 2.4 |
| Strawberry flavor | 1.0 |
| Colloidal silicon dioxide | 1.5 |
| Sodium stearyl fumarate | 1.8 |
| Ferric oxide | 0.2 |

### Example 2

Particles size distribution measurements of the first and second populations of exemplary compositions according to certain embodiments of the disclosure as set forth in Tables 3-4 were performed. The measurements were conducted using Sympatec Helos Rodos laser diffraction system with precision acceptance criteria as set forth in USP <429>. The results are outlined in Tables 5-8 below:

TABLE 5

| | Particle size (μm) | | |
|---|---|---|---|
| | 1^{st} population of particles of a | | |
| Measurement | composition as set forth in Table 3 | | |
| No. | D₁₀ | D₅₀ | D₉₀ |
| 1 | 401.34 | 486.38 | 583.33 |
| 2 | 395.05 | 484.82 | 583.35 |
| 3 | 402.52 | 491.58 | 595.25 |
| 4 | 409.12 | 490.83 | 587.81 |
| 5 | 411.55 | 497.26 | 597.49 |
| 6 | 411.55 | 494.84 | 593.26 |

TABLE 6

| | Particle size (μm) | | |
|---|---|---|---|
| | 2^{nd} population of particles of a | | |
| Measurement | composition as set forth in Table3 | | |
| No. | D₁₀ | D₅₀ | D₉₀ |
| 1 | 24.81 | 144.06 | 383.67 |
| 2 | 22.01 | 131.52 | 333.42 |
| 3 | 22.50 | 137.38 | 361.40 |
| 4 | 21.29 | 131.90 | 326.36 |
| 5 | 22.19 | 140.50 | 351.18 |
| 6 | 24.29 | 144.79 | 403.37 |
| 7 | 22.64 | 138.32 | 351.11 |
| 8 | 23.06 | 143.74 | 400.51 |
| 9 | 23.66 | 145.74 | 397.81 |
| 10 | 22.29 | 135.50 | 404.24 |

TABLE 7

| | Particle size (μm) | | |
|---|---|---|---|
| | 1^{st} population of particles of a | | |
| Measurement | composition as set forth in Table 4 | | |
| No. | D₁₀ | D₅₀ | D₉₀ |
| 1 | 379.59 | 477.58 | 579.98 |
| 2 | 387.43 | 481.46 | 585.06 |
| 3 | 396.55 | 486.40 | 588.68 |

TABLE 8

| | Particle size (μm) | | |
|---|---|---|---|
| | 2^{nd} population of particles of a | | |
| Measurement | composition as set forth in Table4 | | |
| No. | D₁₀ | D₅₀ | D₉₀ |
| 1 | 12.32 | 123.38 | 360.20 |
| 2 | 12.13 | 126.48 | 376.00 |
| 3 | 11.33 | 117.93 | 317.15 |
| 4 | 11.14 | 117.20 | 321.98 |
| 5 | 11.61 | 122.55 | 378.38 |

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. A method of manufacturing an orally disintegrating tablet comprising a plurality of compressed particles having a bimodal size distribution comprising a first and a second population of particles, the method comprising the steps of:
(i) applying a solution or dispersion comprising a drug coating layer to a plurality of inert seeds thereby generating a plurality of cores, wherein the drug coating layer comprises a proton pump inhibitor, an alkalizing agent, a binder, a surfactant, and a filler;
(ii) applying a solution or dispersion comprising a subcoating layer to the plurality of cores thereby generating a plurality of subcoated cores, wherein the subcoating layer comprises a binder, an anti-tacking agent, a surfactant, and a filler;
(iii) applying a solution or dispersion comprising an enteric coating layer to the subcoated cores thereby generating enteric coated cores constituting a first population of particles, wherein the enteric coating layer is the outermost coating layer in said population of particles comprising an enteric polymer, an anti-tacking agent, a plasticizer, a colorant, and optionally an anti-static agent;
(iv) mixing the first population of particles with a second population of particles comprising a mixture of powders comprising a disintegrant and a sugar alcohol; and
(v) compressing the mixture of the first and second populations of particles thereby obtaining the orally disintegrating tablet.

2. The method of claim 1, wherein the weight percent ratio of the first population of particles to the second population of particles is about 0.5:1 to about 1:0.5.

3. The method of claim 1, wherein the inert seeds comprise sugar spheres; the drug coating layer comprises lansoprazole, meglumine, hydroxypropyl methyl cellulose, mannitol, and polysorbate; the subcoating layer comprises hydroxypropyl methyl cellulose, mannitol, talc, and polysorbate; and the enteric coating layer comprises hydroxypropyl methyl cellulose phthalate, cetyl alcohol, triethyl citrate, talc, and titanium dioxide.

4. The method of claim 1, wherein the inert seeds are in an amount of about 2% to about 10%, the drug coating layer is in an amount of about 1% to about 50%, the subcoating layer is in an amount of about 2% to about 30%, and the enteric coating layer is in an amount of about 10% to about 20% by weight of the total mass of the orally disintegrating tablet.

5. The method of claim 1, wherein the disintegrant is in an amount of about 2% to about 20% by weight of the total mass of the orally disintegrating tablet, and wherein the weight percent ratio of the sugar alcohol to the disintegrant is at least 2:1.

6. An orally disintegrating tablet comprising a plurality of compressed particles having a bimodal size distribution comprising a first and a second population of particles, wherein the first population of particles comprises:
a) inert seeds comprising a filler;
b) a drug coating layer over the inert seeds, the drug coating layer comprising a proton pump inhibitor, an alkalizing agent, a binder, a surfactant, and a filler;
c) a subcoating layer over the drug coating layer, the subcoating layer comprising a binder, an anti-tacking agent, a surfactant, and a filler; and
d) an enteric coating over the subcoating layer, the enteric coating comprising an enteric polymer, an anti-tacking agent, a plasticizer, a colorant, and optionally an anti-static agent, wherein the enteric coating layer is the outermost coating layer in said population; and
wherein the second population of particles comprises a mixture of powders comprising a disintegrant and a sugar alcohol.

7. The orally disintegrating tablet of claim 6, wherein the ratio of the median particle size of the first population of particles to the median particle size of the second population of particles is about 2:1 to about 5:1, wherein the first population of particles has a median particle size in the range of about 400 μm to about 600 μm and the second population of particles has a median particle size in the range of about 50 μm to about 250 μm.

8. The orally disintegrating tablet of claim 7, wherein the first population of particles has a d₁₀ particle size in the range of about 300 μm to about 500 μm, and wherein the second population of particles has a d₁₀ particle size in the range of about 1 μm to about 100 μm.

9. The orally disintegrating tablet of claim 7, wherein the first population of particles has a d₉₀ particle size in the range of about 500 μm to about 700 μm, and wherein the second population of particles has a d₉₀ particle size in the range of about 250 μm to about 500 μm.

10. The orally disintegrating tablet of claim 6, wherein the weight percent ratio of the first population of particles to the second population of particles is about 0.5:1 to about 1:0.5.

11. The orally disintegrating tablet of claim 6, wherein the proton pump inhibitor comprises lansoprazole, omeprazole, pantoprazole, leminoprazole, perprazole, rabeprazole, or a pharmaceutically acceptable salt thereof, and a mixture or combination thereof.

12. The orally disintegrating tablet of claim 6, wherein the inert seeds comprise sugar spheres.

13. The orally disintegrating tablet of claim 6, wherein the drug coating layer comprises lansoprazole, meglumine, hydroxypropyl methyl cellulose, mannitol, and polysorbate.

14. The orally disintegrating tablet of claim 6, wherein the subcoating layer comprises hydroxypropyl methyl cellulose, mannitol, talc, and polysorbate.

15. The orally disintegrating tablet of claim 6, wherein the enteric coating layer comprises hydroxypropyl methyl cellulose phthalate, cetyl alcohol, triethyl citrate, talc, and titanium dioxide.

16. The orally disintegrating tablet of claim 6 comprising inert seeds in an amount of about 2% to about 10%, a drug coating layer in an amount of about 1% to about 50%, a subcoating layer in an amount of about 2% to about 30%, and an enteric coating layer in an amount of about 10% to about 20% by weight of the total mass of the orally disintegrating tablet.

17. The orally disintegrating tablet of claim 6, wherein the disintegrant is in an amount of about 2% to about 20% by weight of the total mass of the orally disintegrating tablet.

18. The orally disintegrating tablet of claim 6, wherein the weight percent ratio of the sugar alcohol to the disintegrant is at least 2:1.

19. A method of inhibiting gastric acid secretion, the method comprising administering to a subject in need thereof the orally disintegrating tablet of claim 6.

20. A method of treating a disease or disorder selected from the group consisting of gastroesophageal reflux disease, gastritis, peptic ulcers (duodenal and gastric) and erosive esophagitis, the method comprising administering to a subject in need thereof the orally disintegrating tablet of claim 6.
